(19) United States
(12) Patent Application Publication
(10) Pub. No.: US 2011/0046620 A1
(43) Pub. Date: Feb. 24, 2011

(54) CORDLESS HEATED FORCEPS

(75) Inventors: Simon Lewandowski, Saint Petersburg, FL (US); Jack E. Hunnell, Hillsborough, NC (US)

Correspondence Address: INTELLECTUAL PROPERTY / TECHNOLOGY LAW, PO BOX 14329, RESEARCH TRIANGLE PARK, NC, 27709, US

(73) Assignee: TRIANGLE BIOMEDICAL SCIENCES, INC., Durham, NC (US)

(21) Appl. No.: 12/859,493

(22) Filed: Aug. 19, 2010

Related U.S. Application Data
(60) Provisional application No. 61/235,343, filed on Aug. 19, 2009.

(51) Int. Cl. A61B 18/18

(52) U.S. Cl. 606/41

(57) ABSTRACT

A cordless heated forceps device useful for manipulating tissue samples in a paraffin or other embedding medium, e.g., in histology and pathology labs. The forceps in one implementation has tip elements that are bound to a resistive heat-dissipating component with copper wire and soldered to form a unitary tip assembly. Circuitry can be provided in the body, e.g., the legs, of the forceps device, and may include two circuits, one for resistive heating of the forceps tip elements, and the other for recharging one or more rechargeable batteries in the forceps device. The forceps legs have air gaps or non-conductive material at their distal portions in proximity to the forceps tip elements. The forceps device can be constructed to mate with a recharging base station adapted to connect to a computer or processor with network connectivity, for remote actuation.

### CROSS-REFERENCE TO RELATED APPLICATION

This claims the benefit of priority under 35 USC §119 of U.S. Provisional Patent Application No. 61/235,343, filed on Aug. 19, 2009. The disclosure of the foregoing application is hereby incorporated herein by reference in its entirety, for all purposes.

### FIELD

The present disclosure relates to a cordless forceps device useful for manipulating tissue specimens in paraffin embedding media in histology and pathology laboratories. Aspects of the disclosure also relate to cautery devices or other medical instruments.

### DESCRIPTION OF THE RELATED ART

In histological or pathological analysis of specimens, e.g., patient tissue samples, the specimens are placed in paraffin mounting media so that thin slices of the specimen can be cut and placed on microscope slides for examination. Heated forceps must be utilized in order to locally melt the paraffin in order that the specimens can be manipulated.

The conventional state of the art involves use of mechanical forceps that are placed into a heated receptacle, over an open flame, or on a hot plate surface, to reach a desired temperature that is higher than the melting point of the paraffin. Such forceps have no capability to maintain a constant temperature and therefore lose heat, due to conductive, convective and radiative heat transfer while the forceps are in use. Further, the use of an open flame to repetitively heat the forceps can create a safety hazard.

Alternatively, corded heated forceps have been developed, which are heated by electrical resistance heating structures to achieve a desired temperature. However, when heated corded forceps devices are utilized in environments containing the molten paraffin embedding media, cords can become fouled with paraffin and cause tissue contamination issues, as well as raise general cleanliness and efficiency considerations.

Additionally, existing heated forceps are required to be manually handled, so that any excess heating of forceps tip portions raises issues of safety and comfort in use of devices. For example, corded heated forceps may become progressively heated in a proximal direction by heat conduction, as the forceps is being used. This in turn requires the user to place fingers continually more rearwardly as usage of the forceps proceeds, and a progressively larger portion of the instrument becomes uncomfortably hot to the touch.

Moreover, both mechanical forceps and conventional corded heated forceps require technicians to initiate heating of the forceps and to wait until the forceps reach a desired temperature.

In consequence, the art continues to seek improvements in ease of use, efficiency, and cleanliness of forceps devices and associated wiring for histological applications.

### SUMMARY

The present disclosure relates generally to a rechargeable cordless heated forceps device useful for orienting and manipulating tissue samples in paraffin media, and forceps/charging unit assemblies comprising same.

In one aspect, the disclosure relates to a rechargeable cordless heated forceps device, comprising sections pivotably connected with one another to enable distal portions of the sections comprising tip elements to be translated toward or away from one another, including one or more rechargeable batteries and circuitry for charging said rechargeable batteries and electrically heating the tip elements, and an air gap and/or non-conductive material at a distal portion of each section for cooling of said distal portion.

Another aspect of the disclosure relates to a cordless heated forceps assembly, comprising: (i) a rechargeable cordless heated forceps device as described above, and (ii) a charger base adapted to engage the rechargeable cordless heated forceps device for charging of the rechargeable batteries therein.

Another aspect of the disclosure relates to a charging station that includes charging circuitry coupleable with a power supply and one or more cordless heated forceps devices each including at least one rechargeable battery therein for heating thereof, wherein the charging circuitry is adapted to charge the at least one rechargeable battery in each of the one or more cordless heated forceps devices when the charging station is engaged with the one or more cordless heated forceps devices.

A further aspect of the disclosure relates to a rechargeable cordless heated forceps device, including leg portions pivotally connected to one another, electrically heatable tip elements in the leg portions, and at least one air gap and/or non-conductive material in a distal part of each of the leg portions for cooling of the device.

A still further aspect of the disclosure relates to a method of thermally managing a forceps for manipulating specimens in an embedding medium, comprising fabricating the forceps with distal tips that are resistively heated by one or more rechargeable batteries in the forceps, and controlling rearward heat flow in the forceps by providing an air gap opening and/or non-conductive material in a distal portion of the forceps.

Other aspects, features and embodiments of the disclosure will be more fully apparent from the ensuing description and appended claims.

### BRIEF DESCRIPTION OF THE DRAWINGS

FIG. 1 is a perspective view of a cordless heated forceps according to one embodiment of the present disclosure.

FIG. 2 is a top plan view of a distal portion of a tip portion of a cordless heated forceps of the disclosure.

FIG. 3 is a cordless heated forceps assembly in accordance with another aspect of the disclosure, including a charging base unit and a cordless heated forceps.

FIG. 4 is a multiple cordless heated forceps assembly in accordance with another aspect of the disclosure, including a charging base unit and a plurality of cordless heated forceps engaged in charging relationship thereon.

### DETAILED DESCRIPTION

The present disclosure relates to a cordless heated forceps device useful in histology and pathology labs for manipulating specimens such as patient tissue samples in paraffin media, and to an assembly including one or more forceps device of such type and a charging base therefor.

As used herein, the singular forms “a”, “and”, and “the” include plural referents unless the context clearly dictates otherwise.

The disclosure is set forth herein in various embodiments, and with reference to various features and aspects of the cordless forceps. The disclosure contemplates such features, aspects and embodiments in various permutations and combinations, as being within the scope of the disclosure. The disclosure may therefore be specified as comprising, consisting or consisting essentially of, any of such combinations and permutations of these specific features, aspects and embodiments, or a selected one or ones thereof.

Although the disclosure is hereafter shown and described with reference to cordless heated forceps devices of a type that is useful for example in histology and pathology labs, it will be recognized that the disclosure is not thus limited, but rather extends to and encompasses cordless heated devices used for other purposes, e.g., electrocautery, tissue ablation and other medical applications.

The disclosure in one aspect relates to a rechargeable cordless heated forceps device, comprising sections pivotably connected with one another to enable distal portions of the sections comprising tip elements to be translated toward or away from one another, including one or more rechargeable batteries and circuitry for charging said rechargeable batteries and electrically heating the tip elements, and an air gap and/or non-conductive material at a distal portion of each section for cooling of said distal portion.

Such arrangement permits highly effective thermal management of the cordless heated forceps device. For example, an air gap opening or openings in the distal portion of the heated forceps accommodates convective heat transfer from the heated tip, and thereby avoids excessive heat transfer to the portion of the forceps that is gripped by the user.

The forceps device may be formed so that the sections of the forceps device are coupled to one another in such manner as to constitute legs that are biased to an open position and manually squeezed at an appropriate location to close the forceps by translation of one or both of the legs so that the forceps is closed to accommodate gripping when the manual pressure is applied.

Alternatively, the forceps device may be formed so that the sections of the forceps device are coupled to one another in such manner as to constitute legs that are biased closed and manually squeezed at an appropriate location to open the forceps by translation of one or both of the legs so that the forceps is opened to accommodate gripping when the manual pressure is released.

In one embodiment of such forceps device, each of the tip elements is bound to a resistor with wire in a circular wrap, and soldered so that the solder penetrates interstitially into the circular wrap of wire, thereby forming a unitary tip/resistor assembly. Alternatively, the forceps device may be constructed with each of the tip elements being bound to a themistor with wire in a circular wrap, and soldered so that the solder penetrates interstitially into the circular wrap of wire, thereby forming a unitary tip/thermistor assembly.

The forceps device circuitry may be arranged with suitable circuitry for recharging the rechargeable battery or batteries of the device and electrically heating the tip elements. For example, the circuitry can include two circuits, one of which is arranged for recharging said rechargeable batteries and the other of which is arranged for electrically heating the tip elements. In one implementation, the visual state of charge (SOC) indicator may include an array of indicator lights, each of which is a different color indicative of a state of charge condition. For example, the visual SOC indicator may include an array of red, yellow, and green LEDs, indicating low, moderate and high SOC states, respectively.

The circuitry may also be connected to a visual temperature indicator that is arranged to provide a visual output indicative of the temperature of the tip element(s) of the device. For example, such indicator may include a blue indicator light element correlative with a cool or cold state of the tip element(s), and an orange indicator light element indicating that the tip element(s) are at a desired or set point temperature.

The forceps device may also include circuitry coupled with an on-off switch whereby the rechargeable battery or batteries in the device are controllable for supplying energy to the tip elements for heating thereof.

The forceps device can be formed in any suitable structural arrangement that provides an operable instrument. For example, the device may be formed of sections that are pivotally coupled to open or close upon exertion of manual pressure by a user, and may for example be spring-biased to an open or closed position. The sections may be similarly shaped to one another, e.g., as half-sections that are pivotally coupled to each other, or the sections may include a main body portion to which leg portions are coupled to be pivotally arranged in relation to one another, or the sections may include a main body section including a distally extending leg portion that is pivotally coupled to a second leg section.

Each section of the forceps device may include a housing formed of a plastic material. The sections may be of convergently tapered shape at their distal end portions, and employ finger graps, thermally insulative pads, or other similar elements to enhance the grippability and manipulatability of the forceps device.

The rechargeable cordless heated forceps device of the disclosure thus may include leg portions pivotally connected to one another, with electrically heatable tip elements in the leg portions, and at least one air gap through-opening and/or non-conductive material in a distal part of each of the leg portions for cooling of the device.

The cordless forceps device of the disclosure may be utilized in an assembly, comprising: (i) a rechargeable cordless heated forceps device, e.g., as described above; and (ii) a charger base adapted to cooperate with the rechargeable cordless heated forceps device for charging of the rechargeable batteries in the rechargeable cordless heated forceps device.

A further aspect of the disclosure relates to a charging station that includes charging circuitry coupleable with a power supply and one or more cordless heated forceps devices each including at least one rechargeable battery therein for heating thereof, wherein the charging circuitry is adapted to charge the at least one rechargeable battery in each of the one or more cordless heated forceps devices when the charging station is engaged with the one or more cordless heated forceps devices. For such purpose, the charging station may engage directly or wirelessly or in any other suitable manner with the cordless heated forceps device(s). For example, the charger base may be configured as a charging pad, on which the cordless heated forceps device(s) can be reposed for charging of the rechargeable battery or batteries therein.

In one embodiment, the charger base may be fabricated to have a trapezoidal shape in elevational cross-section, and the charger base may have a generally frustopyramidal shape, so that the cordless heated forceps device(s) can mate with the charger base when the forceps device(s) are in an open position.

In various embodiments, the charger base may be constructed and arranged with charging contacts at specific contact points thereon, e.g., on top, side, bottom or other surfaces thereof that are matably engageable with recharging contacts on the forceps device when the forceps device is engaged with the charger base for charging of the rechargeable batteries. The charger base may have a top surface on which the forceps device is supported when the forceps device is engaged with the charger base, and/or the charger base may include a lower support member with cavities in which the tip elements of the forceps device can be reposed to keep the tip elements at a warm or otherwise desired temperature.

In one embodiment, the charger base may have contacts that mate with contacts on facing surfaces of the forceps legs, so that the forceps contacts mate with a trapezoidal solid portion of the base for engagement with respective base contacts on the base structure.

The charger base may in various embodiments be fabricated to include a USB or ethernet port, or other suitable port or connector structure, for networked connection of the charger base to a digital communications network, internet-accessible hardware, computer or other processor or CPU. The charger base can thereby be arranged for remote actuation. This arrangement enables a pathology or histology worker to actuate the base station remotely before arriving at the laboratory, so that the heated forceps is in a ready-to-go condition at the time such worker arrives at the laboratory for tissue processing work. An on/off switch may be provided on the forceps and/or on the base station, as desired. The on/off feature may be separate from or related to such remote control aspect. The cordless heated forceps assembly may also utilize remote control and other features in the base station to minimize energy usage of the device and to maximize thermal effectiveness of its use.

In one embodiment, the charger base is constructed and arranged to matably engage with a plurality of forceps devices. The base charger and forceps may be arranged with circuitry in the charger base and in the forceps device that cooperatively enable the heating of the forceps device tip elements to elevated temperature, e.g., in a range of 75° C. to 85° C. Such circuitry can be of any suitable type, and may for example include resistors, capacitors, inductors, and other electrical components as discrete componentry of the circuitry, or such electrical components may be provided in integrated circuitry as a chip structure in the forceps device and/or charger base.

The charger base in various embodiments can be arranged to accommodate multiple cordless heated forceps devices with individual docking positions, so that when one heated forceps after reaching a predetermined temperature is removed, a next one is heated to, or maintained at, appropriate temperature. By such arrangement, the first forceps can be returned to the charger base for recharging of the battery, and the second forceps is available in a ready state, so that no loss of operating time is occasioned.

The forceps device may utilize resistors or resistive elements or materials for electrical resistance heating of the tip elements of the device. In one such arrangement, each tip is adjacent to a resistor and wrapped with copper wire in a circular wrap, following which the assembly is soldered, so that the solder penetrates interstitially to produce a unitary tip/resistor assembly having superior thermal characteristics.

The disclosure in another embodiment relates to a rechargeable cordless heated forceps device, including opposedly facing leg portions pivotally connected to one another, electrically heatable tip elements coupled in power-supplying relationship to at least one rechargeable battery, and at least one air gap through-opening and/or non-conductive material in a distal part of each of the leg portions for convective cooling of the device. As used in such context, the term “non-conductive material” refers to a material that is poorly thermally conducting, or thermally insulating, in relation to the other structural material of the forceps device.

The air gap through-openings or non-conducting material in the legs of the forceps device is advantageously arranged so that the forceps is able to be manually handled for extended periods of time without discomfort to the hand of the user.

Thus, in various specific implementations of the disclosure, the cordless heated forceps comprises a forceps body including two hingedly joined legs. The forceps body can be formed of injection-molded or thermoformed plastic or other suitable material. The legs may be symmetrical or asymmetrical in relation to one another, and may be hingedly joined to one another in a spring-biased manner, so as to be biased to an open position, or alternatively, a closed position.

One or more rechargeable batteries, e.g., one or more AAA lithium ion batteries, may be disposed in the cordless heated forceps device, such as in a main body portion of the device, or in one or both leg portions of the device. The hinge of the forceps body can be a simple pin. Internal to the hinge is a spring which maintains the forceps in an open position, or alternatively, a closed position. The forceps tips can be formed of any suitable material, preferably a conductive material such as metal, e.g., stainless steel, chrome-plated brass, or the like, and the forceps tips can be integrally molded into the legs or alternatively can be separately fabricated and subsequently installed.

Each leg has an air gap and/or non-conductive material at the distal portion of the heated forceps tip, to avoid excessive heat transfer to the portion of the forceps that is gripped by the user, so that such portion remains cool. The air gap may be a through-opening in the distal portion of the device in each leg portion. Optionally, an insulation element such as a low thermal conductivity grip or pad can be provided on the finger- and thumb-engaging surfaces of the distal tip portion of the respective legs of the forceps, to maximize user comfort in the utilization of the instrument. The non-conductive material can be of any suitable type, e.g., cork, wood, low conductivity plastic, natural or synthetic fibrous material, or the like.

The forceps device in one embodiment of the disclosure has recharging circuitry contacts on or in the device. Such contacts may be at any suitable location on or in the device, e.g., on an external or internal surface of the forceps, or in a port or plug cavity of the device.

In one embodiment, such contacts are disposed on one or both interior surfaces of the leg portions of the device, and are positioned to mate with contacts on a trapezoidal solid portion of a base station for engagement with respective charging contacts on such base station. In such manner, the recharging circuitry contacts on the forceps device are kept out of contact with the user during normal operation of the forceps device, but such recharging contacts on the interior surfaces of the leg portions of the forceps device engage charging contacts on the base station when the forceps device is docked to charge the rechargeable battery in the forceps device.

In various embodiments of the disclosure, each forceps tip element is adjacent to a resistor or thermistor and wrapped with conductive wire, e.g., with copper wire in a circular or helical wrap, following which the assembly is soldered, so that solder penetrates interstitially to produce a unitary tip/resistor assembly or a unitary tip/thermistor assembly. The tip elements or tip/resistor assemblies or tip/thermistor assemblies may be fabricated to be removable, so that tips of different sizes and shapes can be readily installed and removed from the device, e.g., as the tips begin to wear.

The forceps device may be constructed to contain two separate circuits in the body or one or both of the legs of the forceps device. The forceps tips can be heated through one circuit and the device may be charged by the base station through a second circuit.

It will be apparent from the foregoing that the forceps of the disclosure may be fabricated in any of a wide variety of manners, to provide on-board power to resistively heated tip elements while thermally managing the heat that is generated at the distal tips of the forceps device.

In a method aspect, the disclosure contemplates a method of thermally managing a forceps for manipulating specimens in an embedding medium, comprising fabricating the forceps with distal tips that are resistively heated by one or more rechargeable batteries in the forceps, and controlling rearward heat flow in the forceps by providing an air gap opening and/or non-conductive material in a distal portion of the forceps.

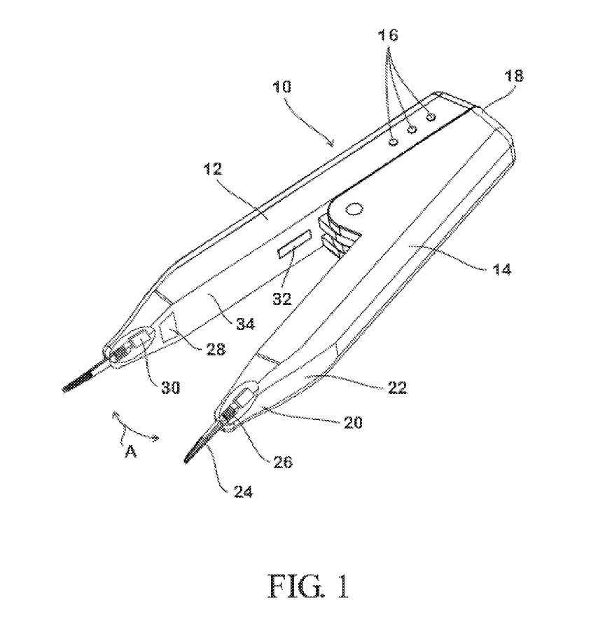

Referring now to the drawings, FIG. 1 is a perspective view of a cordless heated forceps 10 according to one embodiment of the present disclosure. The forceps includes two sections 12 and 14 of elongate form, hingedly connected and/or aligned to one another, and spring-biased to an open position, to accommodate closure of the forceps by exertion of manual pressure on the side surfaces 22 at distal end portions 20 of the device. Alternatively, the forceps device could be constructed to be biased to a closed position, as previously discussed. For such purpose, the sections of the forceps may be fabricated to be pivotally translatable in relation to one another (along the path indicated by arrow A in FIG. 1) by a hinged connection at the proximal end 18 of the device, or a hinged connection at an intermediate position between the proximal end 18 and the distal end portions 20.

The forceps tip elements 24 at the distal end of the forceps device may be associated with a resistor or thermistor for electrical resistance heating of such element, by means of the circumferential wrapping, e.g., spiral or helical or circular wrapping, of wire member 26 about the forceps tip elements, as described more fully hereinafter.

Within the housing of each section 12 and 14 of the forceps device, are deployed circuitry 30, including a heating circuit at the distal part of the sections and a charging circuit at the proximal part of the sections.

Each of the sections at its distal portion has an air opening or non-conductive material 28 therein. With a through opening 28, heat generated in the heating circuitry is dissipated by convection as ambient air passes through the through opening. Such convective heat transfer serves to maintain the distal portion of the forceps in contact with the user's hand and fingers substantially cooler than would otherwise be the case, thereby permitting more comfortable use by the histology technician or other user of the forceps device. Alternatively, with a non-conductive material 28, heat generated in the tip region is not transmitted rearwardly to the areas contacted by the user's hand.

At the proximal end of the forceps is a multi-light array 16, e.g., a three LED array, coupled to charging circuitry in the housing (not shown), to indicate state of charge (SOC) of the rechargeable batteries in the housing of each section of the forceps device. The LEDs may suitably be of red, yellow and green colors, corresponding to low, medium and high SOC. Alternatively, rheostat controlled brightness of a light element or array could be used to indicate instrument operating characteristics such as state of charge. As a further variation in the forceps device, the SOC indicator may comprise a multi-color display element that outputs a specific color to indicate a current state of charge.

Batteries, e.g., two AAA rechargeable batteries, may be accommodated in each section of the forceps device, or otherwise disposed in the body of the device, such as in the leg portions thereof. The housing of the forceps device may be constructed to allow removal and replacement of the rechargeable batteries, when such batteries are at the end of their useful lives.

On a surface 34 of each section of the forceps device is a recharging contact 32 (only one of which is shown in FIG. 1, on the left-hand section in the perspective view shown, it being recognized that the right-hand section is similarly constructed). The interior surface positioning of the recharging contact permits the user of the forceps to avoid contact therewith in the normal operation of the device. Such interior surface location therefore permits the user to avoid fouling of the contact surface with paraffin or other ambient contaminants and to accommodate recharging of the rechargeable batteries in the forceps device. Alternatively, the charging circuitry recharging contacts can be on another surface of the forceps device. The circuitry in the forceps device can be of any appropriate type, and can for example include battery short circuit, over temperature, over charge and under charge protection circuitry.

While interior surface positioning of the contacts is advantageous for the reasons mentioned, it will be appreciated that contacts may be provided anywhere on or in the body of the forceps device, to accommodate recharging action by the charging base hereinafter described.

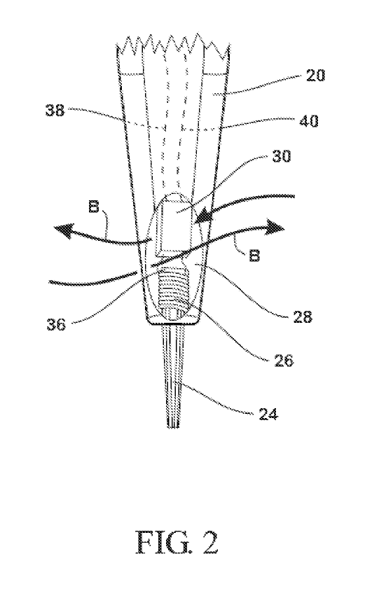

FIG. 2 is a top plan view of a distal portion of a tip portion 20 of a cordless heated forceps of the disclosure, in one embodiment. The distal tip portion 20 in the illustrated embodiment has a through opening 28 therein, thereby accommodating convective cooling of the forceps device (convective air flows being indicated by arrows B in FIG. 2).

In the FIG. 2 embodiment, the distal tip element 24 is associated with a resistive heating element 36. The resistive heating element 36 for such purpose can be abutted against distal tip element 24, to form a distal assembly that is wrapped circumferentially with wire 26, as shown, with the wire being coupled to the heating circuit of the circuitry 30. The wire-wrapped assembly may then be exposed to hot solder, so that the solder flows into the interstices of the windings to form an integrated resistive heating assembly. A thermistor could alternatively be assembled with the tip element of the forceps device, and soldered in the same manner after wire wrapping of the assembly. The heating circuit is connected by wires 38 and 40 to the rechargeable batteries (not shown), whereby the rechargeable batteries supply power to the heating circuit for heating of the distal tip element of the forceps device.

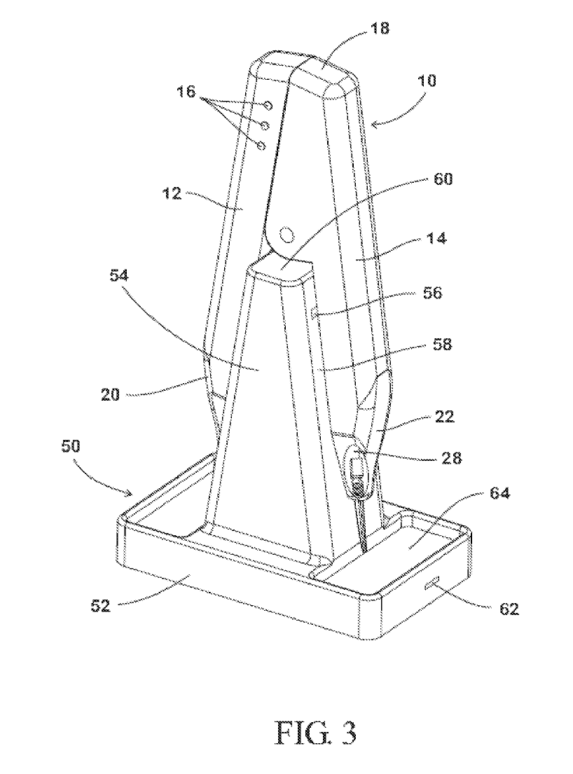

FIG. 3 is a cordless heated forceps assembly in accordance with another aspect of the disclosure, including a charging base unit 50 and a cordless heated forceps 10. The reference numbers in FIG. 3 are numbered correspondingly with respect to the numbering in FIGS. 1 and 2, as regards the same or corresponding elements.

As shown in FIG. 3, the cordless forceps 10 is reposed on the charging base unit 50, with the pivotal hinge portion of the forceps device being in supported contact with upper surface 60 and/or side surfaces of the frustopyramidal shaped charging platform 54. On the sides 58 of the charging platform 54 that are in mating engagement with the forceps device, charging contacts 56 (only one of which is visible in the perspective view of FIG. 3, but the other of which is symmetrically arranged on the opposite face of the charging platform) mate with the recharging contacts on the interior surfaces of the sections of the forceps device, when the forceps device is matably engaged with the recharging platform.

In this manner, current is flowed from the charging base unit 50, through the charging contacts 56 thereof and recharging contacts on the interior surfaces of the respective sections of the forceps device, to charge the rechargeable batteries in the housings of the sections of the forceps device. For this purpose, the charging platform 54 may contain fast charging circuitry, whereby the cordless forceps device may be quickly charged according to a predetermined charging profile, to bring the SOC of the batteries in the forceps device to a desired value in a predetermined time-frame.

The charging base unit 50 may be fabricated with a lower support member 52 defining cavities 64 as illustrated, to accommodate the tip elements of the forceps device therein. The lower support member for such purpose may be formed of a polysulfone or bis-maleimide polymer or other heat-resistant or insulative material, to prevent the hot tip elements of the forceps device from causing any damage when the forceps device is docked with the charging base unit 50. The charging base cavities 64 also function as drip trays for any residual paraffin dripping off the tips of the forceps. These cavities may be equipped with a removable liner, e.g., a disposable liner to facilitate cleaning.

As a further feature, the lower support member 52 may be equipped with a USB, ethernet or other port 62, whereby the charging base unit 50 can be networked with a personal computer, controller or CPU on a digital communications network, by means of suitable networking or remote management software, to enable remote control of the charging base unit. The charging base unit, as well as the forceps device, may be equipped with manual or automatic turn-on and shut-off controls, so that the base charging unit and forceps device can be separately or concurrently actuated for operation or termination of operation.

It will be appreciated that the circuitry of the charging base and the forceps device may be arranged so that the forceps device, when on the charging base, is capable of being charged while the tip elements are simultaneously heated by the charging base so that the forceps device is ready to use without waiting for warm-up of the device.

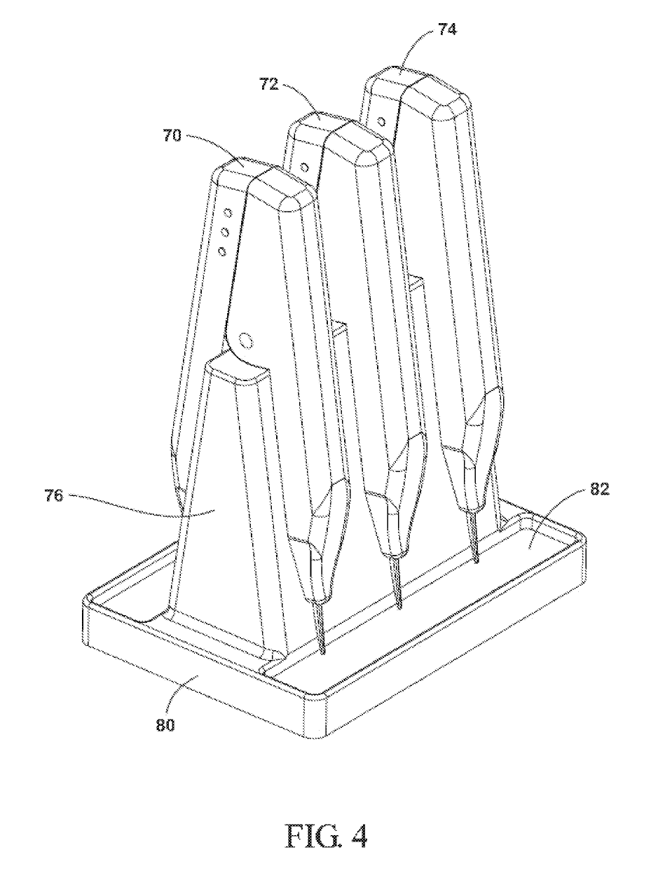

FIG. 4 is a multiple cordless heated forceps assembly in accordance with another aspect of the disclosure, including a charging base unit including frustopyramidal charging platform 76, lower support member 80 defining forceps tip element-receiving cavities 82 therein, and a plurality of cordless heated forceps 70, 72 and 74 engaged in charging relationship thereon, to accommodate multiple users, or single user sequential use of different forceps devices.

It will therefore be apparent that the base charging unit and forceps devices of the disclosure can be constructed and arranged in a variety of ways for operation, to achieve ease of use, quick deployment capability, efficiency, and effective thermal management of cordless heated forceps devices.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. A rechargeable cordless heated forceps device, comprising sections pivotably connected with one another to enable distal portions of the sections comprising tip elements to be translated toward or away from one another, one or more rechargeable batteries and circuitry for charging said rechargeable batteries and electrically heating the tip elements, and an air gap opening and/or non-conductive material at a distal portion of each section.

2. The forceps device of claim 1, wherein each of said tip elements is bound to an electronic component with wire in a circular wrap, and soldered so that the solder penetrates interstitially into the circular wrap of wire, thereby forming a unitary tip/electronic component assembly.

3. The forceps device of claim 1, wherein the circuitry comprises two circuits, one of which is arranged for recharging said rechargeable batteries and the other of which is arranged for electrically heating the tip elements.

4. The forceps device of claim 1, wherein said circuitry is connected to a visual state of charge indicator arranged to indicate the state of charge of the rechargeable batteries in the device.

5. The forceps device of claim 4, wherein the visual state of charge indicator comprises an array of indicator lights, each of which is a different color indicative of a state of charge condition.

6. The forceps device of claim 1, wherein each of said tip elements is bound to a resistor with wire in a circular wrap, and soldered so that the solder penetrates interstitially into the circular wrap of wire, thereby forming a unitary tip/resistor assembly.

7. The forceps device of claim 4, wherein the visual state of charge indicator comprises an indicator light whose degree of illumination is indicative of a state of charge condition

8. The forceps device of claim 1, wherein each section comprises a housing formed of a plastic or non-conductive material.

9. The forceps device of claim 1, wherein the sections are of convergently tapered shape at their distal end portions.

10. A cordless heated forceps assembly, comprising: (i) a rechargeable cordless heated forceps device as claimed in claim 1, and (ii) a charger base adapted to engage the rechargeable cordless heated forceps device for charging of the rechargeable batteries therein.

11. The assembly of claim 10, wherein the charger base has a trapezoidal shape in elevational cross-section.

12. The assembly of claim 10, wherein the charger base has a frustopyramidal shape.

13. The assembly of claim 10, wherein the charger base has charging contacts on side surfaces thereof that are matably engageable with the recharging contacts on the forceps device when the forceps device is engaged with the charger base for charging of the rechargeable batteries.

14. The assembly of claim 10, wherein the charger base has a top surface on which the forceps device is supported when the forceps device is engaged with the charger base.

15. The assembly of claim 10, wherein the charger base includes a lower support member with cavities arranged to receive the tip elements of the forceps device therein.

16. The assembly of claim 10, wherein the charger base includes a USB port or ethernet port for networked connection of the charger base to a digital communications network.

17. The assembly of claim 10, wherein the charger base is arranged to be remotely actuated.

18. The assembly of claim 10, wherein the charger base is constructed and arranged to matably engage with a plurality of forceps devices.

19. The assembly of claim 10, wherein the forceps device is constructed and arranged to heat said tip elements to temperature above a predetermined working temperature.

20. A rechargeable cordless heated forceps device, including leg portions pivotally connected to one another, electrically heatable tip elements in the leg portions, and at least one air gap opening or non-conductive material in a distal part of each of the leg portions.

21. The forceps device of claim 1, further comprising protective circuitry, selected from among battery short circuit, over temperature, over charge and under charge protection circuitry.

22. A rechargeable cordless heated forceps device according to claim 1, comprising an air gap through-opening at a distal portion of each section for cooling of said distal portion.

23. A charging station that includes charging circuitry coupleable with a power supply and one or more cordless heated forceps devices each including at least one rechargeable battery therein for heating thereof, wherein the charging circuitry is adapted to charge the at least one rechargeable battery in each of the one or more cordless heated forceps devices when the charging station is engaged with the one or more cordless heated forceps devices.

24. A method of thermally managing a forceps for manipulating specimens in an embedding medium, comprising fabricating the forceps with distal tips that are resistively heated by one or more rechargeable batteries in the forceps, and controlling rearward heat flow in the forceps by providing an air gap opening and/or non-conductive material in a distal portion of the forceps.
